(19) United States
(12) Patent Application Publication
(10) Pub. No.: US 2016/0243782 A1
(43) Pub. Date: Aug. 25, 2016

(54) METHOD FOR TREATING CELLULOSE-CONTAINING BIOMASS

(71) Applicant: SHOWA DENKO K.K., Minato-ku, Tokyo (JP)

(72) Inventors: Ichiro FUJITA, Tokyo (JP); Shinji YAMAKI, Tokyo (JP); Tadashi YONEDA, Tokyo (JP)

(73) Assignee: SHOWA DENKO K.K., Tokyo (JP)

(21) Appl. No.: 15/027,599

(22) PCT Filed: Sep. 9, 2014
(86) PCT No.: PCT/JP2014/073795

§ 371 Date: Apr. 6, 2016

(30) Foreign Application Priority Data
Oct. 7, 2013 (JP) 2013-210211

(51) Int. Cl. B30B 15/34; C12P 19/02; C12P 19/14; B02C 19/22; B30B 11/24

(52) U.S. Cl. CPC B30B 15/34; B02C 19/22; B30B 11/24; C12P 19/14; C12P 19/02; C12P 2201/00

(57) ABSTRACT

A treatment method for biomass which is a pretreatment method including continuously performing hydrothermal treatment using a screw extruder to produce a biomass composition for saccharification from cellulose-containing biomass serving as a raw material with a small amount of electricity. The treatment method sequentially includes: pulverizing, in a pulverization section of the screw extruder, the cellulose-containing biomass so as to have a maximum grain size of 1,000 μm or less and adjusting a water content ratio thereof to from 30% to 80%; performing, in a heating section of the screw extruder, hydrothermal treatment at a temperature of from 205° C. to 250° C. for 0.1 minute to 10 minutes while performing kneading pulverization having a grinding effect; and cooling, in a cooling section of the screw extruder downstream of the heating section, a treated product to 100° C. or less to recover the treated product.

### TECHNICAL FIELD

The present invention relates to a treatment method for cellulose-containing biomass. More specifically, the present invention relates to a treatment method for cellulose-containing biomass involving continuously performing hydrothermal treatment through use of a screw extruder to produce a biomass composition exhibiting high saccharification performance from the cellulose-containing biomass serving as a raw material with a small amount of electricity, a production method for a biomass composition for saccharification, and a production method for a sugar.

### BACKGROUND ART

As part of measures against global warming, there have been wide investigations on production of various chemical products including ethanol and the like through effective utilization of cellulose-containing biomass. Examples of the cellulose-containing biomass include hard biomass such as cedar or cypress, and soft biomass, such as rice straw, wheat straw, corncobs, cassava, bagasse, or sugar cane leaves. The biomass may contain hemicellulose, lignin, and the like, and hence it is difficult to directly saccharify the biomass. Therefore, there have been proposals to enhance its saccharification performance through various pretreatments.

As a pretreatment method for enhancing the saccharification performance, there have been proposed a method involving adding an acid or alkali and performing hydrothermal treatment, and a method involving a combination of hydrothermal treatment without a chemical and physical pulverization treatment (JP 2006-136263 A; Patent Document 1). Further, in addition to those methods, there have been proposed water vapor blasting, ammonia blasting, ozone oxidation, white-rot fungus treatment, microwave irradiation, electron beam irradiation, and y-ray irradiation (Journal of The Japan Wood Research Society, 53, 1-13 (2007); Non-Patent Document 1).

However, when those pretreatment methods are studied for industrially useful treatment steps, a device and a method capable of continuously and efficiently treating a raw material in a large amount are not specifically disclosed, while those methods each have an effect of enhancing the saccharification performance to some extent.

As a method of continuously and efficiently performing pretreatment prior to enzymatic saccharification of biomass, in JP 59-192093 A (Patent Document 2), and JP 59-192094 A and JP 2012-170355 A (Patent Documents 3 and 4; U.S. Pat. No. 4,642,287), it has been proposed that a pretreatment method involving kneading biomass with an alkali and subjecting the biomass to hydrothermal treatment with a twin-screw extruder can be continuously performed at a high concentration in a short treatment time as compared to conventional fine pulverization treatment and alkali steam treatment.

However, the pretreatment methods disclosed in Patent Documents 2 to 4 each involve high chemical cost owing to the use of the alkali at a ratio of around 20% with respect to a raw material, and inevitably require neutralization and washing of the added alkali prior to the enzymatic saccharification. Therefore, a problem in terms of economy and efficiency including even a saccharification step is not solved. Further, the pretreatment methods of Patent Documents 2 and 3 are each disclosed as substantially a combination of pulverization and alkali steaming, but with regard to the treatment conditions, only the conditions related to the alkali steaming, such as a heating temperature, a heating time, and the amounts of the raw material and the alkali to be loaded, are presented, and the configuration of a device related to the pulverization, etc. are not presented. The embodiments for carrying out those inventions are unclear.

As a method of easily and rapidly performing pretreatment of plant biomass, in JP 2011-130745 A (Patent Document 5), there has been proposed a method involving sequentially performing pretreatment operations in an extruder in a continuous manner, the operations involving adding a decomposer to plant biomass coarsely pulverized into a preset size or less and subjecting the plant biomass to pressurized hot water treatment, and subsequent operations before saccharification loading in which the plant biomass is mixed with an enzyme for saccharification. However, in Patent Document 5, the conditions under which the treatment method is performed, and data on saccharification of the biomass as to what level of saccharification performance is obtained are not presented, while a flowchart and a screw configuration of the extruder for the method are disclosed. The overall performance and efficiency including even a saccharification step are unclear.

In view of the foregoing, there is a demand for establishment of a pretreatment method for cellulose-containing biomass which is high not only in saccharification performance of a treated product but also in overall production efficiency including even sugar production, capable of continuously treating a raw material in a large amount, and industrially useful.

### PRIOR ART

### Patent Document

Patent Document 1: JP 2006-136263 A
Patent Document 2: JP 59-192093 A (U.S. Pat. No. 4,642,287)
Patent Document 3: JP 59-192094 A (U.S. Pat. No. 4,642,287)
Patent Document 4: JP 2012-170355 A
Patent Document 5: JP 2011-130745 A

### Non-Patent Document

Non-Patent Document 1: Journal of the Japan Wood Research Society, 53, 1-13 (2007)

### DISCLOSURE OF INVENTION

### Problem to be Solved by Invention

An object of the present invention is to provide a treatment method for cellulose-containing biomass which is capable of continuously providing a cellulose-containing composition exhibiting high saccharification performance to glucose with a small amount of electricity, and is industrially highly useful, a production method for a cellulose-containing composition for saccharification including conducting the treatment method, and a production method for a sugar including hydrolyzing the cellulose-containing composition for saccharification.

### Means to Solve Problem

The inventors of the present invention have made extensive investigations in order to achieve the above-mentioned object. As a result, the inventors have found that, when, in a treatment method for obtaining a sugar from cellulose-containing biomass, a screw extruder is used, and the cellulose-containing biomass is sequentially subjected to: pulverization and adjustment of a water content ratio in a pulverization section of the device; hydrothermal treatment concurrently with kneading pulverization having a grinding effect in a heating section of the device; and cooling in a cooling section of the device, in a continuous manner, a cellulose-containing composition exhibiting high saccharification performance to glucose is obtained with a small amount of electricity. Thus, the present invention has been completed.

That is, the present invention relates to the following treatment method for cellulose-containing biomass.

[1] A treatment method for biomass, which is a pretreatment method comprising continuously performing hydrothermal treatment through use of a screw extruder to produce a biomass composition for saccharification from cellulose-containing biomass serving as a raw material with a small amount of electricity,
the treatment method sequentially comprising:
pulverizing, in a pulverization section of the screw extruder, the cellulose-containing biomass serving as a raw material so as to have a maximum grain size of 1,000 μm or less and adjusting a water content ratio thereof to from 30% to 80%;
performing, in a heating section of the screw extruder, hydrothermal treatment at a temperature of from 205° C. to 250° C. for 0.1 minute to 10 minutes while performing kneading pulverization having a grinding effect; and
cooling, in a cooling section of the screw extruder downstream of the heating section, a treated product to 100° C. or less to recover the treated.
[2] The treatment method for biomass according to [1] above, in which, in the heating section, the kneading pulverization having a grinding effect is performed concurrently with the hydrothermal treatment by arranging an element including a seal ring and at least one set of a kneading disc and/or a left-hand screw arranged immediately upstream of the seal ring.
[3] The treatment method for biomass according to [1] or [2] above, in which, in the cooling section downstream of the heating section, the cooling is performed by installing a water cooling jacket and/or a liquid feed line.
[4] The treatment method for biomass according to any one of [1] to [3] above, in which the screw extruder is a co-rotating twin-screw extruder.
[5] The treatment method for biomass according to any one of [1] to [4] above, in which the cellulose-containing biomass is soft biomass.
[6] A production method for a biomass composition for saccharification, comprising conducting the treatment method described in any one of [1] to [5] above.
[7] A production method for a sugar, comprising hydrolyzing a biomass composition obtained by the production method described in [6] above.

### Effects of Invention

According to the treatment method for cellulose-containing biomass of the present invention comprising continuously performing the hydrothermal treatment concurrently with the kneading pulverization having a grinding effect through use of a screw extruder, a cellulose-containing composition for saccharification useful as a raw material for producing a sugar through a hydrolysis reaction is obtained with a small amount of electricity, and a sugar can be efficiently produced from the cellulose-containing biomass.

### BRIEF DESCRIPTION OF DRAWINGS

FIG. 1(A) is a configuration view of a screw extruder A used in Examples 7 to 9 and Comparative Examples 2 and 3, FIG. 1(B) is a configuration view of a screw extruder B used in Comparative Example 1, FIG. 1(C) is a configuration view of a screw extruder C used in Examples 1 to 6 and Comparative Examples 4 and 5, FIG. 1(D) is a configuration view of a screw extruder D used in Comparative Example 7, and FIG. 1(E) is a configuration view of a screw extruder E used in Comparative Example 6.

FIG. 2 includes graphs for showing a relationship between a hydrothermal temperature and the power consumption of the extruder motor with respect to the treated raw material in Examples 1 to 9 and Comparative Examples 2 to 5.

FIG. 3 includes graphs for showing a relationship between a hydrothermal temperature and the power consumption of the extruder motor with respect to the produced sugar in Examples 1 to 9 and Comparative Examples 2 to 5.

### MODE FOR CARRYING OUT INVENTION

The present invention is described in detail below. A treatment method of the present invention comprises feeding cellulose-containing biomass to a screw extruder, and continuously performing hydrothermal treatment concurrently with kneading pulverization having a grinding effect. An acid or alkali as an additive may be added to water in a raw material used in the treatment method, but it is industrially preferred to use only water, which is generally available, because the use of the additive not only increases chemical cost but also produces cost for detoxification, such as neutralization, in a subsequent step.

[Cellulose-Containing Biomass]

The biomass used in the treatment method of the present invention means a biopolymer (nucleic acid, protein, or polysaccharide) or an industrial resource derived from such constituent component, other than exhaustible resources (fossil fuel, such as petroleum, coal, or natural gas). Therefore, examples of the cellulose-containing biomass include hard biomass such as wood, and soft biomass such as rice straw, wheat straw, corncobs, cassava, bagasse, or sugar cane leaves. Soft biomass is preferred in consideration of the ease of the pretreatment, and further, bagasse and sugar cane leaves are particularly preferred in consideration of their global storage potential and collection cost.

[Type of Screw Extruder]

The screw extruder to be used in the treatment method of the present invention may be any one of a single-screw extruder, a multi-screw extruder, and a special extruder. Of those, a multi-screw extruder, which can apply stronger shear to a biomass material, is preferred, and a twin-screw extruder is more preferred because of its generality and versatility.

As the multi-screw extruder, there may be adopted any one of a type in which screw shafts are parallel to one another and a conical type in which the screw shafts cross obliquely one another. Of those, a parallel type is preferred.

Any one of an engaged-screw type and a non-engaged-screw type may be adopted, but of those, an engaged-screw type is preferred because of a high kneading effect and many practical examples.

With regard to a screw rotation direction, any one of a co-rotation type and a counter-rotation type may be adopted, but of those, a co-rotation type is preferred because of a self-cleaning effect.

[Raw Material Feed Portion of Screw Extruder]

A hopper to be used to stably feed a raw material to a cylinder of the screw extruder is not limited as long as the hopper has a function capable of generating a feed pressure required for a feed portion of a screw without causing bridging of the raw material, and examples of such hopper include a vibration hopper, a hopper with a force feeder, a hopper dryer, a vacuum hopper, and a nitrogen purge hopper. A hopper comprising a screw located on the inside of the hopper and configured to forcibly push a material into the cylinder is preferred from the viewpoint of stably feeding the raw material.

A device configured to quantitatively feed the raw material to the screw extruder is mounted below the hopper. The quantitative feeding device is not limited as long as the device has a function of enabling quantitative feeding, and examples of such device include a mass feeder and a constant volume feeder. Of those, a mass feeder is preferred in view of feeding raw material biomass, which generally has a low bulk density and non-uniform shapes and sizes. In order to feed the raw material to the screw extruder more securely, it is preferred to mount a compactor configured to forcibly press the raw material into the extruder through use of a screw or a piston so that the bulk density of the material can be increased.

[Cylinder Portion of Screw Extruder]

A cylinder portion of the screw extruder includes the following three sections: a heating section, which is located in the middle portion of the cylinder, and is configured to perform hydrothermal treatment through heating with a heater while grinding the raw material; a pulverization section, which is located upstream of the heating section, and is configured to pulverize the raw material and adjust its water content ratio, to thereby consolidate the material and maintain airtightness; and a cooling section, which is located downstream of the heating section, and is configured to cool the material, to thereby consolidate the material and maintain the airtightness. The screw extruder has an L/D of preferably from 30 to 80, more preferably from 40 to 80, still more preferably from 50 to 80 in its entirety including the pulverization section, the heating section, and the cooling section from the viewpoint of stably maintaining sealing, and performing hydrothermal treatment having an effect of improving the saccharification performance of the raw material biomass. The pulverization section has an L/D of preferably from 10 to 40, more preferably from 10 to 30, still more preferably from 15 to 25. In addition, the heating section has an L/D of preferably from 10 to 65, more preferably from 15 to 60, still more preferably from 20 to 55. The cooling section has an L/D of preferably from 5 to 35, more preferably from 5 to 20, still more preferably from 5 to 10. It should be noted that the “L/D” refers to an effective length represented by a ratio between the length (L) of a screw, which is measured from a start point of screw thread below the hopper to the tip of the screw, and the diameter (D) of the screw.

[Configuration of Pulverization Section of Cylinder]

The pulverization section of the cylinder preferably has a screw configuration in which at least one or more elements each comprising a seal ring and at least one set of a kneading disc (feed kneading disc, neutral kneading disc, or reverse kneading disc) or a left-hand screw arranged upstream of the seal ring (hereinafter abbreviated as “seal ring elements”) are arranged. A state in which the raw material on an upstream side is compressed is achieved by a damming effect exhibited by the arranged seal ring, thereby achieving a state in which the shear force of a screw located upstream of the seal ring is increased. As a result, the raw material is efficiently pulverized and consolidated, and exhibits a function of sealing the pressure of vapor to be generated in the heating section. The pulverization of the raw material is performed not only for achieving the sealing function but also for improving efficiency of the hydrothermal treatment in the heating section. For this, the maximum grain size of the raw material is preferably set to 1,000 μm or less. It should be noted that the maximum grain size is determined through microscopic observation of a sample extracted from the pulverization section immediately upstream of the heating section.

In addition, in the pulverization section, the water content ratio of the raw material is adjusted to preferably from 30 mass % to 80 mass %, more preferably from 30 mass % to 75 mass %, still more preferably from 35 mass % to 70 mass % in order to optimally perform a hydrothermal reaction and achieve an optimal sealing property. The adjustment of the water content ratio may be separately performed before loading, but is preferably performed by installing a liquid feed line in an arbitrary portion of the pulverization section to feed water therethrough with a high-pressure pump from the viewpoint of reducing the number of steps. It should be noted that the water content ratio refers to the ratio of the mass of water to the total mass of the raw material as it is.

[Configuration of Heating Section of Cylinder]

The heating section of the cylinder preferably has a screw configuration in which at least three or more sets of seal ring elements are arranged. The arrangement of a plurality of seal rings in the heating section exhibits such effect that strong grinding stress, which is generated when the cellulose-containing biomass serving as a raw material passes through an extremely narrow clearance between the seal ring and the cylinder, is applied concurrently with the hydrothermal treatment, thereby improving the saccharification performance of cellulose in biomass. For such effect, the clearance between the seal ring and the cylinder is preferably from 0.5% to 10.0%, more preferably from 1.0% to 8.0%, still more preferably from 1.5% to 5.0% with respect to the inner diameter of the cylinder. It should be noted that the inner diameter of the cylinder in a twin-screw extruder refers to the diameter of the cylinder which surrounds one axis of a screw. The heating in the heating section is not limited as long as the cylinder can be heated, but is preferably performed with an electric heater from the viewpoint of temperature controllability. As the conditions of the hydrothermal treatment for the raw material, the temperature of the raw material falls within a range of preferably from 205° C. to 250° C., more preferably from 210° C. to 240° C., still more preferably from 210° C. to 230° C. A time of passage through the heating section falls within a range of preferably from 0.1 minute to 10 minutes, more preferably from 0.2 minute to 9 minutes, still more preferably from 0.3 minute to 7.5 minutes. A pressure in the heating section falls within a range of preferably from 1 MPa to 20 MPa, more preferably from 1.5 MPa to 15 MPa, still more preferably from 2 MPa to 12 MPa.

[Configuration of Cooling Section of Cylinder]

The cooling section of the cylinder preferably includes a water cooling jacket and/or a liquid feed line in order to cool the raw material heated in the heating section. The cooling in the cooling section is performed so that the temperature of the raw material is reduced to preferably 100° C. or less, more preferably 80° C. or less, still more preferably 70° C. or less. With this, vapor generated in the heating section turns into water, and the pressure of vapor flowing downstream together with the treated biomass can be sealed. Further, a pressure regulating valve may be mounted to a discharge port in the cooling section in order to seal the pressure of vapor more stably in the system.

[Pulverization (Adjustment of Grain Size)]

In the treatment method of the present invention, the cellulose-containing biomass serving as a raw material may be directly fed to the screw extruder without pulverization treatment, but is preferably subjected to adjustment of a grain size in advance through coarse pulverization before its feeding. Pulverization means is not particularly limited as long as the means has a function capable of pulverizing a solid substance. For example, the mode of the device may be a dry mode or a wet mode. In addition, the pulverization system of the device may be a batch system or a continuous system. Further, the pulverization force of the device may be provided by any of impact, compression, shearing, friction, and the like.

Preliminary pulverization treatment may be performed with a device which may be used for the pulverization treatment. Specific examples of the device include a coarse crusher, such as a shredder, a jaw crusher, a gyratory crusher, a cutter mill, a cone crusher, a hammer crusher, a roll crusher, or a roll mill; or a medium crusher, such as a stamp mill, an edge runner, a cutting/shearing mill, a rod mill, an autogenous mill, or a roller mill. Of those, a cutter mill is preferred from the viewpoints of a treatment amount and a pulverization range. The time for treating the raw material is not particularly limited as long as the raw material can be homogeneously and finely pulverized by the treatment.

The grain size of the raw material subjected to pulverization in advance before its feeding is preferably a size passing through a screen (sieve) having a screen diameter of from 0.5 mm to 30 mm because, when the discharge screen diameter of the pulverization device is excessively large, the grain size of the cellulose-containing biomass increases, resulting in high sugar production cost owing to a reduction in subsequent pretreatment effects, and when the screen diameter is excessively small, pulverization cost increases. The grain size is more preferably a size passing through a screen of from 1 mm to 30 mm, most preferably a size passing through a screen of from 3 mm to 30 mm. In addition, also in the case of performing pulverization without using a screen, it is preferred to pulverize the raw material so as to achieve a size corresponding to that of a pulverized product in the case of using the screen.

[Adjustment of Water Content Ratio]

In the treatment method of the present invention, the cellulose-containing biomass serving as a raw material may be subjected to adjustment of a water content ratio in advance before its feeding to the screw extruder. As a method of adjusting a water content ratio, there are given addition of water, dewatering, and drying, in accordance with the water content ratio of the raw material before the adjustment. As described above, the water content ratio of the raw material is preferably adjusted to from 30 mass % to 80 mass % in order to optimally perform the hydrothermal reaction and achieve an optimal sealing property.

A biomass composition for saccharification can be efficiently produced by treating the biomass by the above-mentioned method. Further, when the biomass composition for saccharification produced by the above-mentioned method is hydrolyzed, a sugar can be efficiently produced.

### EXAMPLES

The present invention is hereinafter described by way of Examples and Comparative Examples. However, the present invention is by no means limited to the descriptions of Examples and Comparative Examples.

In Examples and Comparative Examples, screw extruders under five kinds of device conditions were used, and cellulose-containing biomass was treated with changing the conditions of a grain size of the raw material, a hydrothermal temperature and a hydrothermal time. Further, a treated sample was evaluated for a saccharification rate, and a method and conditions with less power consumption with respect to the produced sugar, which is excellent in overall performance and efficiency including even a saccharification step, were examined.

[Screw Extruder A]

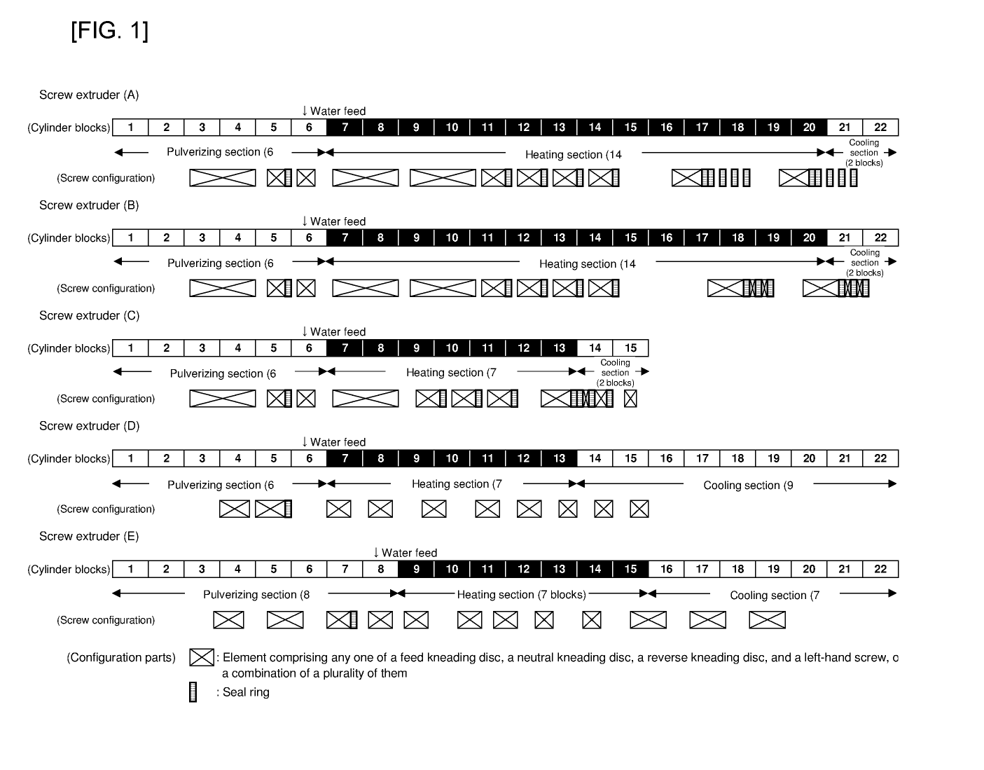

A screw extruder A in which a twin-screw extruder having an L/D of 77.0 and a screw diameter of 32 mm (trade name: TEX30α, manufactured by The Japan Steel Works, Ltd.) was allowed to have the following screw configuration illustrated in FIG. 1(A): a pulverization section in 6 blocks, a heating section in 14 blocks (6 sets of seal ring elements), and a cooling section in 2 blocks was used in Examples 7 to 9 and Comparative Examples 2 and 3.

[Screw Extruders B to E]

Screw extruders B to E (illustrated in FIG. 1(B) to FIG. 1(E), respectively) each having the same configuration as that of the screw extruder A except that the number of blocks of the pulverization section, the number of blocks of the heating section and the number of seal ring elements in the heating section, and the number of blocks of the cooling section were changed as shown in Table 1 were used in Examples and Comparative Examples shown in Table 1.

It should be noted that an L/D value, the number of blocks of sections other than the heating section, the number of seal ring elements in these sections, the number of seal rings used, and a clearance between a seal ring and a screw were also shown in Table 1. In addition, an extruder having a cylinder diameter of 47 mm (trade name: TEX44α, manufactured by The Japan Steel Works, Ltd.) was used as the screw extruder C.

TABLE 1

| | L/D | | | | Number of blocks | | | | Number of seal rings used | | | |
|---|---|---|---|---|---|---|---|---|---|---|---|---|
| | | | Heat- | Cool- | | Heat- | Cool- | | | Heat- | Cool- | |
| | | Pulveri- | ing | ing | Pulveri- | ing | ing | | Pulveri- | ing | ing | |
| Extruder | Whole | zation | sec- | sec- | zation | sec- | sec- | | zation | sec- | sec- | |
| No. | device | section | tion | tion | section | tion | tion | Total | section | tion | tion | Total |
| Screw | 77 | 21 | 49 | 7 | 6 | 14 | 2 | 22 | 1 | 11 | 3 | 15 |
| extruder A | | | | | | | | | | | | |
| Screw | 77 | 21 | 49 | 7 | 6 | 14 | 2 | 22 | 1 | 7 | 3 | 11 |
| extruder B | | | | | | | | | | | | |
| Screw | 52.5 | 21 | 24.5 | 7 | 6 | 7 | 2 | 15 | 1 | 4 | 3 | 8 |
| extruder C | | | | | | | | | | | | |
| Screw | 77 | 21 | 24.5 | 31.5 | 6 | 7 | 9 | 22 | 1 | 0 | 0 | 1 |
| extruder D | | | | | | | | | | | | |
| Screw | 77 | 28 | 24.5 | 24.5 | 8 | 7 | 7 | 22 | 1 | 0 | 0 | 1 |
| extruder E | | | | | | | | | | | | |

| | Number of seal ring elements | | | | | | | |
|---|---|---|---|---|---|---|---|---|
| | | Heat- | Cool- | | Clearance between | | Diameter | |
| | Pulveri- | ing | ing | | seal ring and screw | | of | Examples or |
| Extruder | zation | sec- | sec- | | Length | | cylinder | Comparative |
| No. | section | tion | tion | Total | (mm) | Ratio (%) | (mm) | Examples used |
| Screw | 1 | 6 | 0 | 7 | 0.5 | 1.6 | φ32 | Examples 7-9 |
| extruder A | | | | | | | | Comparative |
| | | | | | | | | Examples 2 and 3 |
| Screw | 1 | 5 | 1 | 7 | 0.5 | 1.6 | φ32 | Comparative |
| extruder B | | | | | | | | Example 1 |
| Screw | 1 | 4 | 3 | 8 | 2.0 | 4.3 | φ47 | Examples 1-6 |
| extruder C | | | | | | | | Comparative |
| | | | | | | | | Examples 4 and 5 |
| Screw | 1 | 0 | 0 | 1 | 0.5 | 1.6 | φ32 | Comparative |
| extruder D | | | | | | | | Example 7 |
| Screw | 1 | 0 | 0 | 1 | 0.5 | 1.6 | φ32 | Comparative |
| extruder E | | | | | | | | Example 6 |

[Preparation of Raw Material Bagasse]

Bagasse was used as the cellulose-containing biomass serving as a raw material.

Bagasse subjected to no treatment (water content ratio: 50%, content ratio of cellulose: 38%, hereinafter abbreviated as “untreated bagasse”), and bagasse (water content ratio: 10.0%, content ratio of cellulose: 42%, hereinafter abbreviated as “3-mm bagasse”) prepared by pulverizing air-dried bagasse with a cutter mill having a screen diameter of 3 mm (MKCM-3, manufactured by Masuko Sangyo Co., Ltd.) were used as the bagasse.

[Analysis Method for Content Ratios of Main Components of Biomass]

The content ratio of cellulose, the content ratio of hemicellulose, and the total content ratio of lignin and an ash content in the biomass were determined by an analysis method (Technical Report NREL/TP-510-42618) of The National Renewable Energy Laboratory (NREL).

[High-Performance Liquid Chromatography Analysis Method and Calculation Method for Content Ratio of Cellulose]

A guard column (KS-G manufactured by Showa Denko K.K.) and a separation column (KS-802 manufactured by Showa Denko K.K.) were connected to each other, and the column temperature was set to 75° C. Pure water was supplied as an eluting solution at a rate of 0.5 ml/min, and a separated component was subjected to quantitative determination with a differential refractive index detector. Thus, the concentration of glucose was determined, and the content ratio of cellulose was calculated based on the following equation.

Content ratio of cellulose (%)={mass of filtrate (g)×(concentration of glucose (%)/100)×0.9}/mass of weighed biomass (g)×100

(The numerical value “0.9” in the equation is a coefficient for correcting changes in molecular weight caused by hydrolysis of cellulose.)

[Calculation Method for Recovery Rate of Cellulose in Pretreatment]

The recovery rate of cellulose from raw material biomass in pretreatment was calculated based on the following equation.

Recovery rate of cellulose (%)={dry mass of hydrothermally treated product (g) after washing with water/dry mass of raw material biomass (g)}×{content ratio of cellulose (%) in hydrothermally treated product/content ratio of cellulose (%) in raw material biomass}×100

[Measurement of Saccharification Performance with Enzyme]

### Preparation of Acid Buffer Solution:

30 g of acetic acid was put in a 100 ml measuring flask, and diluted with pure water to give a 3 M acetic acid aqueous solution. 41 g of sodium acetate was put in a 100 ml measuring flask, and diluted with pure water to give a 3 M sodium acetate aqueous solution. The 5 M acetic acid aqueous solution was added to the 3 M sodium acetate aqueous solution until the pH became 5.0. Thus, an acetic acid buffer solution was obtained.

Preparation of Enzyme Solution:

1.5 g of Meicelase (trademark, cellulase manufactured by Meiji Seika Kaisha, Ltd. (currently Meiji Seika Pharma Co., Ltd.)) was dissolved in 98.5 g of pure water.

The FPU activity (Filter Paper Assay for Saccharifying Cellulase) of the enzyme solution was 6 FPU/g, which was determined according to an analysis method of International Union of Pure and Applied Chemistry (IUPAC) (Pure & Appl. Chem., Vol. 59, No. 2, pp. 257-268, 1987).

Saccharification Reaction:

A rotor was put in a 50 ml glass vessel with a cover, and a composition subjected to pretreatment was weighed so that the amount of cellulose was 0.5 g. Then, 0.6 g of the acetic acid buffer solution and 1.03 g of the enzyme solution were added thereto, and further, pure water was added thereto to give a total of 10 g. The resultant was subjected to a saccharification reaction with the enzyme in a thermostat bath at 40° C. for 72 hours (Hr) while being stirred. The resultant saccharified solution was subjected to quantitative determination for glucose by high-performance liquid chromatography analysis. Thus, a saccharification rate and a sugar utilization rate were calculated by the following equations.

Saccharification rate (%)={concentration of glucose (%) in reaction solution×0.9}/concentration of cellulose (%) in reaction solution at the beginning of reaction}×100

(The numerical value “0.9” in the equation is a coefficient for correcting changes in molecular weight caused by hydrolysis of cellulose.)

Sugar utilization rate (%)=recovery rate of cellulose (%)×saccharification rate (%)/100

[Power Consumption of Extruder Motor]

As power consumption of the extruder motor at the time of hydrothermal treatment, power consumption (kWh) with respect to the treated raw material (kg) (hereinafter referred to as “motor power consumption with respect to the raw material”) and power consumption (kWh) with respect to the produced sugar (kg) obtained by the saccharification of the treated sample (hereinafter referred to as “motor power consumption with respect to sugar”) were calculated by the following equations.

Motor power consumption with respect to the raw material (kWh/kg)=Motor power consumption (kW) at the time of treatment/raw material feed rate in terms of dry mass (kg/Hr) at the time of treatment

Motor power consumption with respect to sugar (kWh/kg)=Motor power consumption with respect to the raw material (kWh/kg)×0.9×{1/[concentration of cellulose (%) in the raw material/100]/[sugar utilization rate (%)/100]}

(The numerical value “0.9” in the equation is a coefficient for correcting changes in molecular weight caused by hydrolysis of cellulose.)

### Example 1

The untreated bagasse was loaded into the screw extruder C having a screw rotation number of 200 rpm with a mass feeder and a compactor at a feed rate of 20.0 kg/Hr of a mass of the bagasse as it was and 10.0 kg/Hr in terms of dry mass. In the pulverization section, water was added through a liquid feed line at a feed rate of 5.0 kg/Hr, and a water content ratio was adjusted so that a water content ratio of 60 mass % (solid content concentration: 40 mass %) was continuously obtained in the pulverization section upstream of the heating section. The hydrothermal treatment was conducted at a temperature of 210° C. and a pressure of 5 MPa, and then the raw material was cooled to 70° C. or less in the cooling section with a water cooling jacket and water provided through a liquid feed line at a feed rate of 5 kg/Hr, followed by recovery of a sample from a discharge port. The hydrothermal time under such conditions was 2.5 minutes. 100 g of water, which was three times the amount of a solid content in the sample, was added to 100 g of the treated sample recovered, and the resultant was suspended, followed by centrifugal filtration with a centrifugal filter (H-122, manufactured by Kokusan Co., Ltd., filter cloth: cotton) at 3,000 rpm. Thus, a water-containing solid content was obtained. The obtained water-containing solid content was calculated for a recovery rate of cellulose and a saccharification rate by the above-mentioned methods.

### Examples 2 to 9 and Comparative Examples 1 to 7

The operations were conducted in the same manner as in Example 1 except that the conditions were changed as shown in Table 2. Table 2 also shows the recovery rate of cellulose, saccharification rate, and sugar utilization rate.

TABLE 2

| | | | | | | | | Water content |
|---|---|---|---|---|---|---|---|---|
| | | | | | | | | ratio in |
| | | Feed rate | | | | Number of | Water feed | pulverization |
| | | | In terms | Screw | | seal ring | rate through | section |
| | | Mass | of dry | rotation | | elements in | liquid feed | (solid content |
| | Kind of | as it is | mass | number | Screw | the heating | line | concentration) |
| | bagasse | [kg/Hr] | [kg/Hr] | [rpm] | extruder | section | [kg/Hr] | [mass %] |
| Example 1 | Untreated | 20.0 | 10.0 | 200 | C | 4 | 5.0 | 60 (40) |
| | bagasse | | | | | | | |
| Example 2 | Untreated | 20.0 | 10.0 | 200 | C | 4 | 5.0 | 60 (40) |
| | bagasse | | | | | | | |
| Example 3 | Untreated | 20.0 | 10.0 | 200 | C | 4 | 5.0 | 60 (40) |
| | bagasse | | | | | | | |
| Example 4 | Untreated | 40.0 | 20.0 | 200 | C | 4 | 10.0 | 60 (40) |
| | bagasse | | | | | | | |
| Example 5 | Untreated | 40.0 | 20.0 | 200 | C | 4 | 10.0 | 60 (40) |
| | bagasse | | | | | | | |
| Example 6 | Untreate | 40.0 | 20.0 | 200 | C | 4 | 10.0 | 60 (40) |
| | bagasse | | | | | | | |
| Comparative | 3-mm | 5.0 | 4.5 | 350 | B | 5 | 4.8 | 54 (46) |
| Example 1 | bagasse | | | | | | | |
| Comparative | 3-mm | 5.0 | 4.5 | 350 | A | 6 | 3.6 | 48 (52) |
| Example 2 | bagasse | | | | | | | |
| Comparative | 3-mm | 5.0 | 4.5 | 350 | A | 6 | 3.6 | 48 (52) |
| Example 3 | bagasse | | | | | | | |
| Example 7 | 3-mm | 5.0 | 4.5 | 350 | A | 6 | 3.6 | 48 (52) |
| | bagasse | | | | | | | |
| Example 8 | 3-mm | 5.0 | 4.5 | 350 | A | 6 | 3.6 | 48 (52) |
| | bagasse | | | | | | | |
| Example 9 | 3-mm | 5.0 | 4.5 | 350 | A | 6 | 3.6 | 48 (52) |
| | bagasse | | | | | | | |
| Comparative | Untreated | 20.0 | 10.0 | 200 | C | 4 | 5.0 | 60 (40) |
| Example 4 | bagasse | | | | | | | |
| Comparative | Untreated | 20.0 | 10.0 | 200 | C | 4 | 5.0 | 60 (40) |
| Example 5 | bagasse | | | | | | | |
| Comparative | 3-mm | 5.0 | 4.5 | 350 | E | 0 | 4.8 | 54 (46) |
| Example 6 | bagasse | | | | | | | |
| Comparative | 3-mm | 5.0 | 4.5 | 350 | D | 0 | 3.6 | 48 (52) |
| Example 7 | bagasse | | | | | | | |

| | | | | | Water in an |
|---|---|---|---|---|---|
| | Maximum | | | | amount |
| | grain size | | | | three times |
| | immediately | Hydrothermal treatment | | | the amount |
| | upstream of | conditions | | | of solid |
| | heating | | | Hydrothermal | content in |
| | section | Temperature | Pressure | time | sample |
| | [μm] | [° C.] | [MPa] | [min] | [g] |
| Example 1 | <1000 | 210 | 5 | 2.5 | 100 |
| Example 2 | <1000 | 220 | 5 | 2.5 | 100 |
| Example 3 | <1000 | 230 | 5 | 2.5 | 100 |
| Example 4 | <1000 | 210 | 5 | 1.2 | 100 |
| Example 5 | <1000 | 220 | 5 | 1.2 | 100 |
| Example 6 | <1000 | 230 | 5 | 1.2 | 100 |
| Comparative | <1000 | 175 | 5 | 7.5 | 138 |
| Example 1 | | | | | |
| Comparative | <1000 | 190 | 5 | 7.5 | 156 |
| Example 2 | | | | | |
| Comparative | <1000 | 200 | 5 | 7.5 | 156 |
| Example 3 | | | | | |
| Example 7 | <1000 | 210 | 5 | 7.5 | 156 |
| Example 8 | <1000 | 220 | 5 | 7.5 | 156 |
| Example 9 | <1000 | 230 | 5 | 7.5 | 156 |
| Comparative | <1000 | 185 | 5 | 2.5 | 120 |
| Example 4 | | | | | |
| Comparative | <1000 | 200 | 5 | 2.5 | 120 |
| Example 5 | | | | | |
| Comparative | <1000 | 175 | 5 | 7.5 | 138 |
| Example 6 | | | | | |
| Comparative | <1000 | 190 | 5 | 7.5 | 156 |
| Example 7 | | | | | |

The results (power consumption of the extruder motor (kW), power consumption of the motor with respect to the raw material (kWh/kg), power consumption of the motor with respect to sugar, recovery rate of cellulose, and saccharification rate) of Examples 1 to 9 and Comparative Examples 1 to 7 are shown in Table 3.

TABLE 3

| | Results of the treatment | | | | |
|---|---|---|---|---|---|
| | | Motor power | | | Motor power |
| | Motor power | consumption with | Cellulose | | consumption |
| | consumption | respect to the raw | recovery | Saccharification | with respect to |
| | (kW) | material (kWh/kg) | rate | rate | sugar (kWh/kg) |
| Example 1 | 10.1 | 1.0 | 93% | 62% | 4.1 |
| Example 2 | 9.8 | 1.0 | 93% | 55% | 4.5 |
| Example 3 | 9.3 | 0.9 | 92% | 52% | 4.6 |
| Example 4 | 14.7 | 0.7 | 95% | 59% | 3.1 |
| Example 5 | 14.7 | 0.7 | 95% | 68% | 2.7 |
| Example 6 | 14.2 | 0.7 | 95% | 56% | 3.2 |
| Comparative | 10.0 | 2.2 | 96% | 66% | 7.5 |
| Example 1 | | | | | |
| Comparative | 12.3 | 2.7 | 92% | 74% | 8.6 |
| Example 2 | | | | | |
| Comparative | 12.0 | 2.7 | 92% | 67% | 9.3 |
| Example 3 | | | | | |
| Example 7 | 8.1 | 1.8 | 90% | 60% | 7.1 |
| Example 8 | 7.9 | 1.8 | 87% | 59% | 7.3 |
| Comparative | 7.8 | 1.7 | 87% | 44% | 9.7 |
| Example 9 | | | | | |
| Comparative | 13.4 | 1.3 | 93% | 50% | 6.8 |
| Example 4 | | | | | |
| Comparative | 13.0 | 1.3 | 94% | 60% | 5.5 |
| Example 5 | | | | | |
| Comparative | 9.0 | 2.0 | 95% | 30% | 15.0 |
| Example 6 | | | | | |
| Comparative | 8.2 | 1.8 | 93% | 34% | 12.2 |
| Example 7 | | | | | |

[Conditions of Extruder in Extrusion Hydrothermal Treatment]

The hydrothermal treatment was performed under the same hydrothermal conditions at a temperature of 175° C. and in time of 7.5 minutes in Comparative Examples 1 and 6, and at a temperature of 190° C. and in time of 7.5 minutes in Comparative Examples 2 and 7, respectively, with an extruder having a different screw configuration. The extrusion hydrothermal treatment in Comparative Examples 1 and 2 was performed with screw extruder B (FIG. 1(B)) in which 5 sets of elements each including a seal ring and at least one set of a kneading disc and/or a left-hand screw arranged immediately upstream of the seal ring (seal ring elements) were arranged in the heating section and with screw extruder A (FIG. 1(A)) in which 6 sets of elements are arranged. In contrast, in Comparative Examples 6 and 7, the extrusion hydrothermal treatment was performed with the screw extruders E and D (FIG. 1(E) and FIG. 1(D)) in which the seal ring element was not arranged in the heating section.

The power consumption with respect to the raw material in each case under different conditions was 2.2 kWh/kg in Comparative Example 1, 2.7 kWh/kg in Comparative Example 2, 2.0 kWh/kg in Comparative Example 6, and 1.8 kWh/kg in Comparative Example 7, and lower in Comparative Examples in 6 and 7 in which the seal ring element was not arranged.

The recovery rate of cellulose after the hydrothermal treatment exceeded 90% in any case, but slightly higher under the condition of the lower hydrothermal treatment temperature of 175° C.

The saccharification rate of the treated sample under the hydrothermal treatment conditions at 175° C. in 7.5 minutes was 66% in Comparative Example 1 including 5 sets of the seal ring elements, while 30% in Comparative Example 6 including 0 set of the seal ring element. The saccharification rate of the treated sample under the hydrothermal treatment conditions at 190° C. in 7.5 minutes was 74% in Comparative Example 2 including 6 sets of the seal ring elements, while 34% in Comparative Example 7 including 0 set of the seal ring element, and it was confirmed that the saccharification rate significantly reduces when no seal ring element is arranged.

The power consumption with respect to sugar varies depending on the results of the recovery rate of cellulose and the saccharification rate. Here, being particularly affected by the saccharification rate, the power consumption with respect to sugar was 7.5 kWh/kg in Comparative Example 1, 8.6 kWh/kg in Comparative Example 2, 15.0 kWh/kg in Comparative Example 6, and 12.2 kWh/kg in Comparative Example 7, and was lower in Comparative Examples 1 and 2 in which the seal ring elements were arranged.

Such effect exhibited by the seal ring element is presumed to be caused as follows: high grinding stress is applied to the cellulose-containing biomass serving as a raw material when it passes through an extremely narrow clearance portion between the seal ring and the cylinder concurrently with the hydrothermal treatment, and thus the saccharification performance of cellulose in the biomass is improved.

From the above-mentioned results, it was confirmed that as device conditions of a screw extruder for obtaining a cellulose-containing composition exhibiting a high saccharification rate with a small amount of electricity, arrangement of a plurality of seal ring elements in the heating section was effective.

[Coarse Pulverization in the Pulverization Section Before the Hydrothermal Treatment]

Examples and Comparative Examples in this description were test examples in which the hydrothermal treatment was able to be performed with a screw extruder. The maximum grain sizes before the extrusion hydrothermal treatment were each determined through microscopic observation of the sample extracted from the pulverization section immediately upstream of the heating section. The maximum grain sizes of the samples were found to be each 1,000 μm or less.

Meanwhile, a test was performed, but the hydrothermal treatment was not able to be actually performed under the following conditions: i.e. the hydrothermal treatment with a screw extruder, in which the seal ring was removed from the seal ring elements in each of the pulverization sections of the screw extruders A and E. The hydrothermal treatment was attempted by supplying 3-mm bagasse to the screw extruders, but the pressure of vapor was not able to be maintained on an upstream side, and a backward flow of the vapor was generated. The treatment failed. In those cases, the maximum grain sizes of the raw material collected from the pulverization section before the hydrothermal treatment each exceeded 1,000 μm, and the pulverization was not sufficient.

From the above-mentioned results, it is presumed that, in the extrusion hydrothermal treatment, the coarse pulverization in the pulverization section exhibits an effect of improving the saccharification performance and an effect of sealing vapor to be generated in the hydrothermal section on an upstream side. It was confirmed that the coarse pulverization was preferably performed to such degree that the maximum grain size fell below 1,000 μm.

[Operation Results in Terms of Conditions of Cooling Section]

Examples and Comparative Examples in this description were test examples in which the hydrothermal treatment was able to be performed with a screw extruder. In each screw extruder, a cooling system of a water cooling jacket and a liquid feed line was used in the cooling section, and a pressure regulating valve was mounted to a discharge port. In the test examples, the temperature in the cooling section was reduced to 100° C. or less through cooling with a water cooling jacket in Examples 7 to 9 and Comparative Examples 1 to 7, and through cooling with the water cooling jacket and water feeding from a liquid feed line to the biomass to be treated in combination in Examples 1 to 6. The hydrothermal treatment was able to be stably and continuously performed in all the test examples.

Meanwhile, a test was performed, but the hydrothermal treatment was not able to be actually performed under the following conditions. When the treatment was performed without using any cooling system of a water cooling jacket and a liquid feed line in each screw extruder, the temperature in the cooling section kept a high temperature state exceeding 100° C., a sample intermittently jetted together with vapor, and the treatment was not able to be stably performed in the case of each screw extruder. As described above, it was confirmed that, for stable operation, the temperature in the cooling section needed to be reduced to 100° C. or less through use of the cooling system of a water cooling jacket and/or a liquid feed line.

[Comparison of Hydrothermal Treatment Temperature]

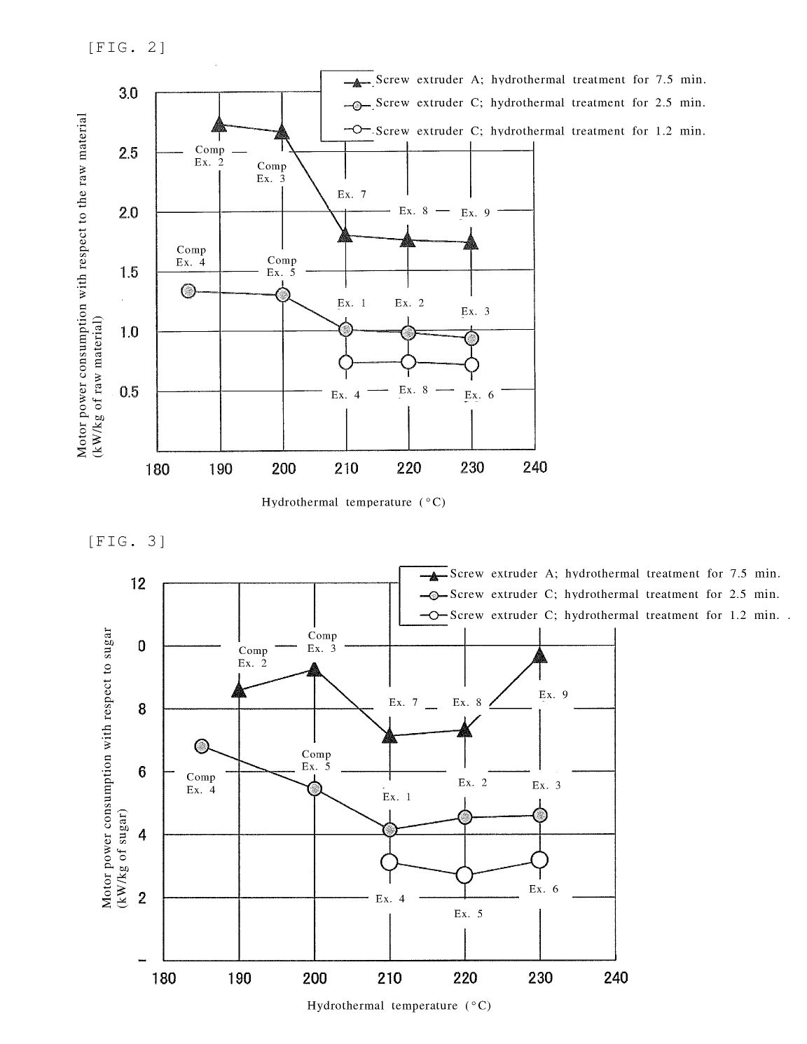

To compare the behavior depending on the hydrothermal treatment temperature, the hydrothermal treatment was conducted under the condition of the hydrothermal time of 7.5 minutes with screw extruder A in Examples 7 to 9 and Comparative Examples 2 and 3, and under the condition of the hydrothermal time of 2.5 minutes with screw extruder C in Examples 1 to 3 and Comparative Examples 4 and 5 by changing the hydrothermal temperature only. The results are shown in Table 2, and FIGS. 2 and 3.

In the hydrothermal treatment with a screw extruder of the present invention, the data such as power consumption and saccharification rate change under the influence of the difference in the raw material conditions, device conditions and hydrothermal treatment conditions. Therefore, the dots of experimental examples under the same conditions except for the temperature are connected with a solid line in FIG. 2 and FIG. 3 and the experimental examples on the same solid line were evaluated for the changes in the power consumption with respect to the raw material and power consumption with respect to sugar due to the difference in the temperature. Further, the examples on different solid lines were evaluated for the changes in the power consumption with respect to the raw material and power consumption with respect to sugar due to the conditions other than the temperature.

As can be seen from the relationship between the hydrothermal temperature and the power consumption with respect to the raw material in FIG. 2, the absolute values of the power consumption with respect to the raw material were generally lower in the hydrothermal treatment in 2.5 minutes with screw extruder C having a smaller L/D (L/D=52.5) compared to the hydrothermal treatment in 7.5 minutes with screw extruder A having a larger L/D (L/D=77.0). The power consumption with respect to the raw material in relation to the treatment temperature had tendency to decrease at the higher temperature and an inflection point, at which the power consumption with respect to the raw material significantly drops, was confirmed in a hydrothermal temperature zone of from 200° C. to 210° C.

The power consumption of the motor in the extrusion hydrothermal treatment, in which kneading including grinding and pulverization with a screw is conducted at the same time of the hydrothermal treatment, depends on the properties of the raw materials at the time of the treatment. Therefore, it is assumed that the above-mentioned inflection point is caused by the improvement in the fluidity of the raw material because most of hemicellulose and lignin as being a non-cellulose component contained in the bagasse as a raw material are solubilized to thereby have a low viscosity at a temperature of from 200° C. to 210° C.

The recovery rate of cellulose after the hydrothermal treatment was around 90% in any case, but generally higher in the case of the hydrothermal time of 2.5 minutes compared to the case of the hydrothermal time of 7.5 minutes, and the rate decreased as the increase in the hydrothermal temperature. From this, it was confirmed that the recovery rate of cellulose has a tendency to decrease with the increase in the thermal history. Further, among the saccharification rate in each of the examples, the maximum of 74% was attained in the hydrothermal treatment in the hydrothermal time of 7.5 minutes at 190° C. (Comparative Example 2) and the maximum of 62% was attained in the hydrothermal treatment in the hydrothermal time of 2.5 minutes at 210° C. (Example 1). Thus, the power consumption with respect to sugar as a result of the recovery rate of cellulose and the saccharification rate was lowest around the temperature of from 200° C. to 210° C.

With respect to the influence due to the difference between “3-mm bagasse” and “untreated bagasse”, when the other conditions are the same, the power consumption increases in the case of using the untreated bagasse due to the increase in the load in the pulverization section, while the saccharification rate nearly unchanges. Along with this, both of the power consumption with respect to the raw material and the power with respect to sugar have a tendency to increase in the case of using the untreated bagasse.

In FIG. 2 and FIG. 3, the power consumption is higher in Examples 7 to 9 and Comparative Examples 2 and 3 using the 3-mm bagasse compared to Examples 1 to 6 and Comparative Examples 4 and 5 using untreated bagasse. Therefore, it can be said that the difference between the 3-mm bagasse and untreated bagasse has relatively less impact and the difference in other conditions has greater influence.

[Hydrothermal Treatment Time]

In Examples 4 to 6, each of the hydrothermal treatment in Examples 1 to 3 at a hydrothermal temperature of 210° C. or higher in 2.5 minutes, which was efficient with a low power consumption with respect to sugar, was conducted by reducing the hydrothermal time to 1.2 minutes. The hydrothermal time was reduced by increasing the feeding rate of the raw material and reducing the retention time in the heating section in a cylinder. As a result, although the power consumption increased by the increase in the feeding rate, the power consumption with respect to the raw material was reduced compared to that in the hydrothermal treatment in 2.5 minutes.

The recovery rate of cellulose after the hydrothermal treatment was 95% in any of examples, which was higher than that in the hydrothermal treatment in 2.5 minutes, and had the same tendency as described above.

The saccharification rate reduced at 210° C. (Example 4), and increased at 220° C. (Example 5) and 210° C. (Example 6). It is presumed that the saccharification rate has an optimal hydrothermal time range at each temperature; and that when the hydrothermal time is shorter than a hydrothermal time in the optimal range, the action of the pretreatment on the biomass is weak, resulting in a reduction in saccharification rate, and when the hydrothermal time is longer than the hydrothermal time in the optimal range, impurities are altered or an excessively decomposed product of a sugar and the like increase, resulting in a reduction in saccharification rate. It was suggested that the optimum hydrothermal time is around 2.5 minutes in the hydrothermal treatment at 210° C., and around 1.2 minutes in the hydrothermal treatment at 220° C. and 230° C. under the conditions in the examples.

As a result, the power consumption with respect to sugar reflecting the recovery rate of cellulose and the saccharification rate was lower in any of Examples 4 to 6 compared to that in the treatment in the hydrothermal time of 2.5 minutes, and the minimum value was 2.7 kWh/kg at 220° C. (Example 5).

### INDUSTRIAL APPLICABILITY

According to the treatment method for biomass of the present invention, an industrially useful composition exhibiting high saccharification performance in saccharification of cellulose-containing biomass can be efficiently obtained at a high concentration in a short time with a small amount of electricity.

1. A treatment method for biomass, which is a pretreatment method comprising continuously performing hydrothermal treatment through use of a screw extruder to produce a biomass composition for saccharification from cellulose-containing biomass serving as a raw material with a small amount of electricity,
the treatment method sequentially comprising:
pulverizing, in a pulverization section of the screw extruder, the cellulose-containing biomass serving as a raw material so as to have a maximum grain size of 1,000 μm or less and adjusting a water content ratio thereof to from 30% to 80%;
performing, in a heating section of the screw extruder, hydrothermal treatment at a temperature of from 205° C. to 250° C. for 0.1 minute to 10 minutes while performing kneading pulverization having a grinding effect; and
cooling, in a cooling section of the screw extruder downstream of the heating section, a treated product to 100° C. or less to recover the treated product.

2. The treatment method for biomass as claimed in claim 1, in which, in the heating section, the kneading pulverization having a grinding effect is performed concurrently with the hydrothermal treatment by arranging an element including a seal ring and at least one set of a kneading disc and/or a left-hand screw arranged immediately upstream of the seal ring.

3. The treatment method for biomass as claimed in claim 1, in which, in the cooling section downstream of the heating section, the cooling is performed by installing a water cooling jacket and/or a liquid feed line.

4. The treatment method for biomass as claimed in claim 1, in which the screw extruder is a co-rotating twin-screw extruder.

5. The treatment method for biomass as claimed in claim 1, in which the cellulose-containing biomass is soft biomass.

6. A production method for a biomass composition for saccharification, comprising conducting the treatment method as claimed in claim 1.

7. A production method for a sugar, comprising hydrolyzing a biomass composition obtained by the production method as claimed in claim 6.
